(19) United States
(12) Patent Application Publication
(10) Pub. No.: US 2020/0079915 A1
(43) Pub. Date: Mar. 12, 2020

(54) POLYMER GEL FILM CONTAINING LIQUID CRYSTAL DROPLETS FOR DETECTING MERCURIC IONS IN WATER AND PREPARATION METHOD THEREOF

(71) Applicant: TAMKANG UNIVERSITY, New Taipei City (TW)

(72) Inventors: CHIH-HSIN CHEN, New Taipei City (TW); JHIH-WEI HUANG, Tainan City (TW); JUNG-JUNG CHANG, Taipei City (TW)

(21) Appl. No.: 16/508,735

(22) Filed: Jul. 11, 2019

(30) Foreign Application Priority Data
Sep. 10, 2018 (TW) 107131730

(51) Int. Cl. C08J 3/075; C08L 5/12; G01N 33/18

(52) U.S. Cl. CPC C08J 3/075; C08L 5/12; C08J 2305/12; C08L 2205/12; C08L 2203/16; G01N 33/1813

(57) ABSTRACT

The invention relates to a method for preparing a polymer gel film containing liquid crystal droplets, comprising the steps of: preparing liquid crystal droplets containing a ligand, by adding a nematic liquid crystal containing a ligand to an aqueous surfactant solution, and mixing, to obtain the liquid crystal droplets containing a ligand; adding the liquid crystal droplets containing a ligand to an aqueous polymer solution, and mixing, to obtain an aqueous polymer solution containing the liquid crystal droplets; and spreading the aqueous polymer solution containing the liquid crystal droplets flatly in a polymer container, and allowing the aqueous polymer solution containing the liquid crystal droplets to gelatinize, to obtain a polymer gel film containing liquid crystal droplets. The invention utilizes liquid crystal droplets dispersed in the agarose to detect mercuric ions in the water, and through the configuration change of the liquid crystal droplets, the mercuric ions is specifically detected.

### CROSS REFERENCE TO RELATED APPLICATION

This application also claims priority to Taiwan Patent Application No. 107131730 filed in the Taiwan Patent Office on Sep. 10, 2018, the entire content of which is incorporated herein by reference.

### BACKGROUND

### Technical Field

The present disclosure relates to a liquid crystal sensor system for detecting heavy metals and more particularly to a polymer gel film containing liquid crystal droplets, which serves to detect mercuric ions in the water by using liquid crystal droplets dispersed in the agarose.

### Related Art

In recent years, with the development of science and technology, pollution has become more and more serious, and more requirements have been raised for the quality of life. Therefore, the demand for detection of chemical molecules that endanger environmental and human health is increasing.

At present, most of the heavy metal detections rely on expensive instruments, often requiring personnel with specialized knowledge to operate and pretreated samples, which are less convenient. The development of liquid crystal sensors has overcome the aforementioned shortcomings. The most notable feature of the liquid crystal sensor is that a user can observe the change of the signal by taking advantage of the color change under normal light without professional training, and the sensor is portable. Relevant objects that can be tested by liquid crystal sensors include metal ions, proteins, amino acids, urea, bacteria, pesticides, DNA, and enzymes.

However, the liquid crystal sensor system currently used for heavy metal detection suffers from a problem that the original position of the liquid crystal droplets cannot be fixed when a test solution (a solution containing a heavy metal) is added, so the configuration changes of the same liquid crystal droplets cannot be observed precisely, and aggregation tends to occur to cause the instability of liquid crystal droplets.

In view of this, there is a need to provide a polymer gel film containing liquid crystal droplets, which solves the above problems.

### SUMMARY

In view of the problem above, the present invention provides a polymer gel film containing liquid crystal droplets, and more particularly to a method for detecting mercuric ions in water by using liquid crystal droplets dispersed in the agarose, with which the problem of incapability of observing the configuration changes of liquid crystal droplets at the same position can be solved.

To achieve the above object, the present invention discloses a method for preparing a polymer gel film containing liquid crystal droplets, which comprises the steps of: preparing liquid crystal droplets containing a ligand, by adding a nematic liquid crystal containing a ligand to an aqueous surfactant solution, and mixing for 10-30 sec by agitating at a rotation speed of 3000 rpm, to obtain a solution of the liquid crystal droplets containing a ligand suspended in an aqueous solution; adding the solution of the liquid crystal droplets containing a ligand to an aqueous polymer solution, and mixing by agitating at a rotation speed of 3000 rpm, to obtain an aqueous polymer solution containing the liquid crystal droplets, wherein the ratio of the solution of the liquid crystal droplets containing a ligand to the aqueous polymer solution is 1:1; and spreading the aqueous polymer solution containing the liquid crystal droplets flatly in a polymer container, and allowing the aqueous polymer solution containing the liquid crystal droplets to gelatinize for 10-15 min, to obtain a polymer gel film containing liquid crystal droplets.

Another object of the present invention is to provide a polymer gel film containing liquid crystal droplets, which is prepared by the method for preparing a polymer gel film containing liquid crystal droplets.

Another object of the present invention is to provide a method for detecting mercuric ions in an aqueous solution by using the polymer gel film containing liquid crystal droplets, which comprises: taking 10-30 μL of a test solution; applying the test solution to the polymer gel film containing liquid crystal droplets; and locating the polymer gel film containing liquid crystal droplets applied with the test solution under a cross-polarized microscope, and observing the conformation inside the liquid crystal droplets.

The present invention mainly has the following effects: 1. specificity for mercuric ions; 2. instant detection for samples, and simple and convenient detection method; and 3. stability (where the detection capability of liquid crystal droplets in agarose can be maintained for three months or more) and reversibility (reusability).

### BRIEF DESCRIPTION OF THE DRAWINGS

FIG. 1 is a flow chart of a method for preparing a polymer gel film containing liquid crystal droplets;

FIG. 2 is a schematic view showing the configuration of liquid crystal droplets of the present invention;

FIG. 3a is a schematic view of an agarose container of the present invention;

FIG. 3b is a schematic view of an agarose film containing liquid crystal droplets of the present invention;

FIG. 4 is a cross-sectional view of a detection component of an ADLC system of the present invention;

FIG. 5 is a FTIR spectrum of an agarose film containing liquid crystal droplets of the present invention;

FIG. 6 is a schematic view under microscope of the agarose fiber containing liquid crystal droplets of the present invention;

FIG. 7 shows the configuration change of liquid crystal droplets in ADLC containing ZT of the present invention;

FIG. 8 shows the analysis of selectivity of the ADLC system of the present invention;

FIG. 9 shows the analysis of inference by metals with ADLC of the present invention;

FIG. 10 shows the analysis of detection limits for Hg²⁺ by ADLC in the presence of different concentrations of agarose in the present invention;

FIG. 11 shows the analysis of reuse rate of the ADLC system of the present invention; and

FIG. 12 is a diagram showing the configuration change of liquid crystal droplets in the ADLC system against Hg²⁺ over long time of detection according to the present invention.

### DETAILED DESCRIPTION

The present invention will be described with reference to preferred embodiments or examples of the present invention, which however are not intended to limit the scope of the present invention. Equal changes and modifications made according to, or without departing from the scope of the present invention are covered by the invention.

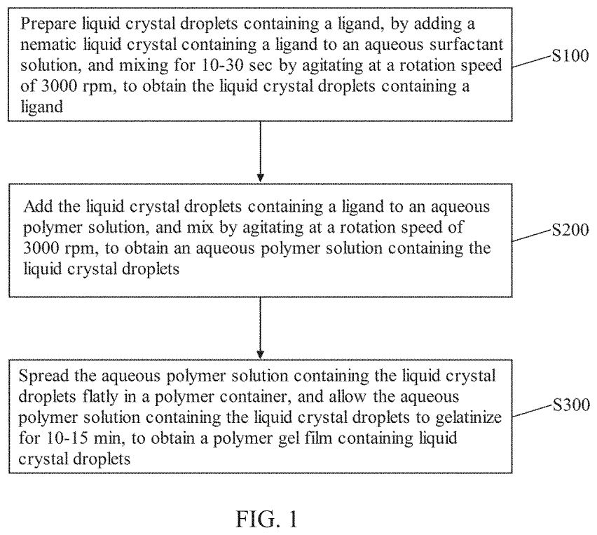

FIG. 1 is a flow chart of a method for preparing a polymer gel film containing liquid crystal droplets. The method comprises the steps of: preparing liquid crystal droplets containing a ligand, by adding a nematic liquid crystal containing a ligand to an aqueous surfactant solution, and mixing for 10-30 sec by agitating at a rotation speed of 3000 rpm, to obtain a solution of the liquid crystal droplets containing a ligand (S100); adding the solution of the liquid crystal droplets containing a ligand to an aqueous polymer solution, and mixing by agitating at a rotation speed of 3000 rpm, to obtain an aqueous polymer solution containing the liquid crystal droplets (S200), wherein the ratio of the solution of the liquid crystal droplets containing a ligand to the aqueous polymer solution is 1:1; and spreading the aqueous polymer solution containing the liquid crystal droplets flatly in a polymer container, and allowing the aqueous polymer solution containing the liquid crystal droplets to gelatinize for 10-15 min, to obtain a polymer gel film containing liquid crystal droplets (S300).

In the method for preparing a polymer gel film containing liquid crystal droplets, the ratio of the aqueous surfactant solution to the nematic liquid crystal containing a ligand is in the range of 100:1-120:1.

The nematic liquid crystal containing a ligand is prepared through a process comprising: dissolving a ligand powder in a nematic liquid crystal to formulate a nematic liquid crystal containing 0.3-0.5% of the ligand. The ligand is 5-(pyridine-4-yl)-2-(5-(pyridin-4-yl)-thiophen-2-yl)thiazole (ZT), a ligand having specificity for mercuric ions.

As described above, the nematic liquid crystal is 4-pentyl-4′-cyanobiphenyl (5CB) having a liquid crystal phase temperature in the range of 23.5° C. to 35° C.

The aqueous surfactant solution is prepared through a process comprising: dissolving a cationic emulsifier powder in ultrapure water to prepare a mother liquor having a concentration of 1%; and then diluting the mother liquor into various concentrations of aqueous surfactant solutions, including 0.03%, 0.05%, and 0.005%.

As described above, the cationic emulsifier is selected from a group consisting of hexadecyltrimethylammonium bromide (C₁₆TAB), tetradecyltrimethylammonium bromide (C₁₄TAB), dodecyltrimethylammonium bromide (C₁₂TAB), and decyltrimethylammonium bromide (C₁₀TAB).

Moreover, the aqueous polymer solution is prepared through a process comprising: dissolving a polymer powder in ultrapure water; and heating to 80° C. to completely dissolve the polymer powder, to obtain an aqueous polymer solution, wherein the concentration of the aqueous polymer solution was 1-2%. The aqueous polymer solution is an aqueous agarose solution or aqueous chitosan solution.

The aqueous agarose solution used in the present invention is also called aqueous agarose. Agarose is considered to be a green material, which is non-toxic, biocompatible and biodegradable. Agarose is hydrophilic and uncharged, will not cause denaturation of sensitive biomacromolecules, and is an ideal inert carrier. In previous studies, based on the measurement of absorbance, agarose gel has been used as a substrate for developing optical sensors for various purposes. However, agarose has not currently been used to develop liquid crystal based sensors.

Another object of the present invention is to provide a polymer gel film containing liquid crystal droplets, which is prepared by the method for preparing a polymer gel film containing liquid crystal droplets.

Further, another object of the present invention is to provide a method for detecting mercuric ions in an aqueous solution by using the polymer gel film containing liquid crystal droplets, which comprises: taking 10-30 μL of a test solution; applying the test solution to the polymer gel film containing liquid crystal droplets; and locating the polymer gel film containing liquid crystal droplets applied with the test liquid under a cross-polarized microscope, and observing the conformation inside the liquid crystal droplets. Preferably, 20 μL of the test liquid is recommended, and the test liquid may be tap water, pond water, river water, and sea water.

In summary, in the method for preparing a polymer gel film containing liquid crystal droplets provided by the present invention, liquid crystal droplets are dispersed in the agarose for detecting mercuric ions in an aqueous solution. After the liquid crystal droplets are added to heated agarose, agarose is gelatinized after cooling to room temperature, whereby the liquid crystal droplets are immobilized in the pores of agarose gel film. When an aqueous solution containing mercuric ions is dropped into the agarose film, the ligand 5-(pyridine-4-yl)-2-(5-(pyridin-4-yl)-thiophen-2-yl)thiazole (ZT) in the liquid crystal droplets complexes with the mercuric ions, causing the configuration of the liquid crystal droplets to change from radial to irregular. This configuration change can be easily observed by the naked eye.

### EXAMPLES

Preparation of detection component of ADLC system

Agarose Dispersed Liquid Crystals system serves to detect mercuric ions in the water by using liquid crystal droplets containing ZT dispersed in agarose. The system is referred to as ADLC system hereinafter, and the detection component of ADLC system is a polymer gel film containing liquid crystal droplets provided in the present invention.

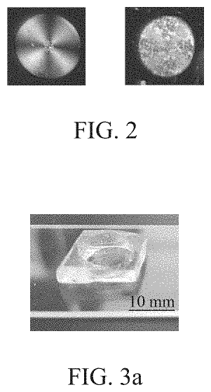

FIG. 2 is a schematic view showing the configuration of liquid crystal droplets of the present invention. A polymer gel film containing liquid crystal droplets provided in the present invention comprises a ligand 5-(pyridine-4-yl)-2-(5-(pyridin-4-yl)thiophen-2-yl)thiazole (ZT) having selectivity for mercuric ions doped in a nematic liquid crystal (4-pentyl-4′-cyanobiphenyl, 5CB). The ZT consists of two molecules of pyridine, one molecule of thiazole, and one molecule of thiophene, and the liquid crystal droplets are stabilized with 0.005% (w/w) of a surfactant that is hexadecyltrimethylammonium bromide (C₁₆TAB). The configuration of the liquid crystal droplets is such that the direction of the liquid crystal droplets is difficult to change and is radial (as showed in the left panel of FIG. 2) due to the protection by agarose. The complex formed when Hg²⁺ binds to ZT will be precipitated on the liquid crystal droplets, causing the formation of an irregular surface image. This irregular configuration is known to be irregular (as showed in the right panel of FIG. 2).

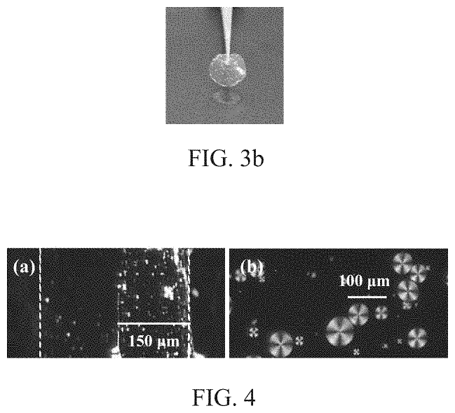

FIG. 3a is a schematic view of an agarose container of the present invention; and FIG. 3b is a schematic view of an agarose film containing liquid crystal droplets of the present invention. First, an aqueous agarose solution was prepared as follows. 1 g of agarose powder was dissolved in 75 mL of ultrapure water, and heated to 80° C. to dissolve the agarose powder completely, to prepare a 1.33% (w/w) aqueous agarose solution for use. Then, 650 μL of the aqueous agarose solution was spread flatly onto a scale pan of 30 mm*30 mm, and allowed to stand for 15 sec to slightly dry the agarose gel. A 1 mL micropipette tip was used as a model. The end with a large opening was gently positioned on the surface of the agarose gel, and 2000 μL of the aqueous agarose solution was added, and allowed to stand for 1 hr. Then the micropipette tip was gently pulled out, and an agarose container was prepared, as shown in FIG. 3a. Further, an agarose gel film containing liquid crystal droplets was prepared. First, 5 μL of ZC-doped 5CB was pipetted to 600 μL of different concentrations of surfactants, and mixed for 15 sec by agitation with a shaker at 3000 rpm, to prepare liquid crystal droplets containing ZT. The ZC-doped 5CB was a 0.3% solution in liquid crystal prepared by dissolving 5-(pyridine-4-yl)-2-(5-(pyridin-4-yl)thiophen-2-yl)thiazole (ZT) powder in 4-pentyl-4′-cyanobiphenyl (5CB). The different concentrations of surfactants were prepared by dissolving hexadecyltrimethylammonium bromide (C₁₆TAB) powder, and tetradecyltrimethylammonium bromide (C₁₄TAB) powder respectively in ultrapure water, to prepare a 1% (w/w) C₁₆TAB solution and a 1% (w/w) C₁₄TAB solution which are used as mother liquors. Then, individual mother liquors were respectively diluted into different concentrations of surfactants, including 0.03%, 0.05%, and 0.005% (w/w) aqueous C₁₆TAB solutions and aqueous C₁₄TAB solutions. Finally, 300 μL of the solution containing liquid crystal droplets was added to 300 μL of the agarose solution at 80° C., and mixed for 2 sec by agitation with a shaker at 3000 rpm. Then, 5 μL of the aqueous agarose solution containing liquid crystal droplets was immediately pipetted and spread flatly in the agaro se gel container, and allowed to stand for 10 min to obtain the detection component of the ADLC system,. The ADLC film was cut off, as shown in FIG. 3b.

FIG. 4 shows dimensions of a detection component of an ADLC system of the present invention, in which (a) shows the dimension of agarose film containing liquid crystal droplets of the ADLC system, and (b) shows the dimension of liquid crystal droplets in ADLC. In order to confirm that liquid crystal droplets containing ZT are indeed embedded in agarose, the thickness of the agarose film and the size of the pores after lyophilization were determined in the present invention, and the properties of the film after being stood to full dryness was measured by an infrared spectrometer. In determination of the film thickness, first, the cross section of the film was placed on a glass slide, and photographed under a microscope at a magnification of 10×, to find that the thickness of the agarose film containing the liquid crystal droplets was about 150 μm. In addition, liquid crystal droplets in radial configuration in the agarose were carefully observed, found to be about 10 to 100 μm in size, confirming that the liquid crystal droplets were indeed embedded in the agarose.

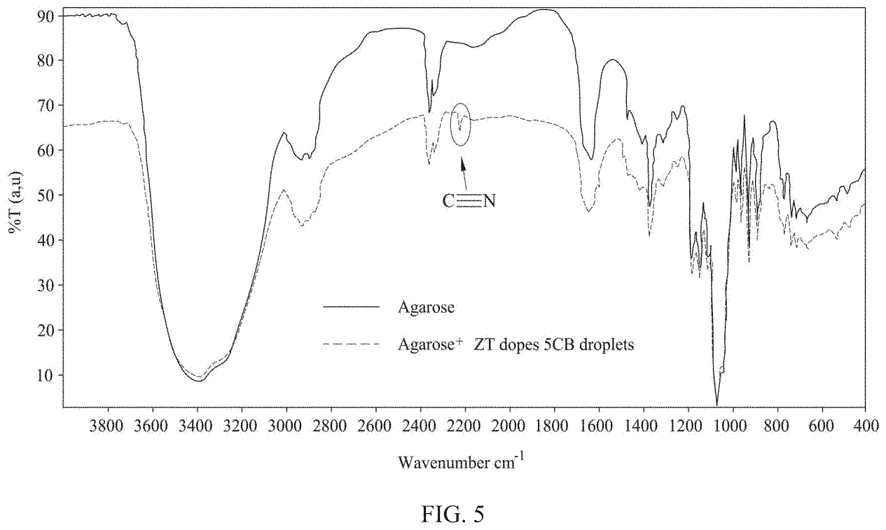

Finally, it was verified by infrared spectroscopy that liquid crystal droplets containing ZT were indeed embedded in agarose. FIG. 5 is a FTIR spectrum of an agarose film containing liquid crystal droplets of the present invention, in which (a) shows an agarose film without liquid crystal droplets, and (b) shows an agarose film containing liquid crystal droplets. It was found by infrared spectroscopy that the ADLC film had stretching vibration of the CN bond at 2227 cm⁻¹, which proved that the liquid crystal was present in the ADLC film.

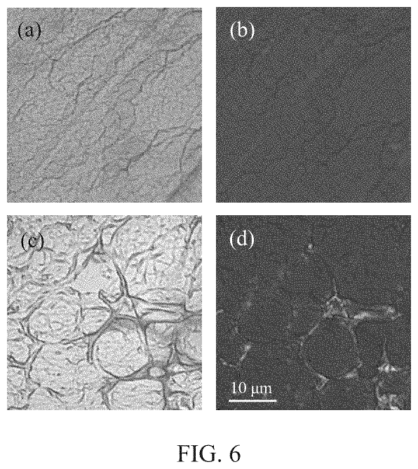

FIG. 6 is a schematic view under microscope of the agarose fiber containing liquid crystal droplets of the present invention, in which (a) shows an agarose fiber without liquid crystal droplets under non-polarized light, (b) shows an agarose fiber without liquid crystal droplets under polarized light, (c) shows an agarose fiber containing liquid crystal droplets under non-polarized light, and (d) shows an agarose fiber containing liquid crystal droplets under polarized light. In order to measure the pore size of the agarose film, in the present invention, agarose films containing and containing no liquid crystal droplets were lyophilized, and photographed under a microscope at a magnification of 20×, to find that the agarose fiber containing no liquid crystal droplets is thin and has no obvious large pores, and the agarose film fiber containing liquid crystal droplets is thick and has obvious aggregation of branches to produce large pores. When observed under polarized light, there are residual liquid crystals at the edges of the pores in the agarose film fibers containing liquid crystal droplets, confirming that before lyophilization, the pore does hold the liquid crystal droplets. After lyophilization, the liquid crystal may be evacuated and therefore there are liquid crystal remaining on the edge of the pores.

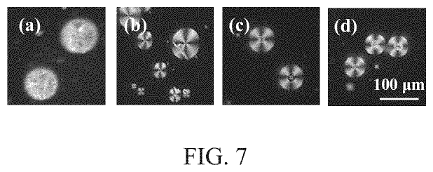

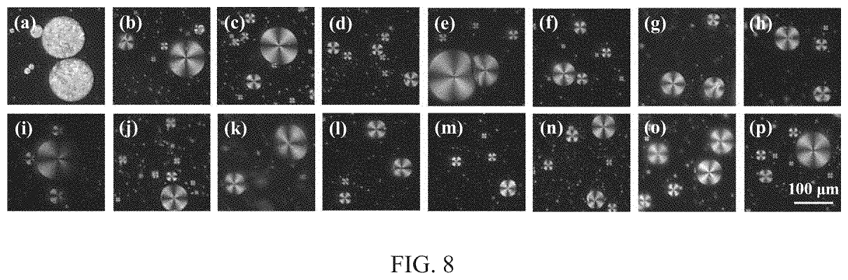

Further, in order to determine that the configuration change of the liquid crystal droplets containing ZT in the ADLC can be effected by adding Hg²⁺, in the present invention, the confirmation is conducted with following controlled experiments. FIG. 7 shows the configuration change of liquid crystal droplets in the ADLC containing ZT of the present invention, in which (a) shows ADLC containing ZT in the presence of 1 mM Hg²⁺, (b) shows ADLC containing ZT in the presence of 0 mM Hg²⁺, (c) shows ADLC containing no ZT in the presence of 1 mM Hg²⁺, and (d) shows ADLC containing ZT in the presence of 0 mM Hg²⁺. The experiments show that when liquid crystal droplets without ZT are dispersed in agarose, the configuration is radial without change regardless of whether the added solution contains mercuric ions or not; and when ZT-containing liquid crystal droplets are dispersed in agarose, the configuration changes can only be observed in the presence of a solution containing Hg²⁺. These suggest that ZT can bind to Hg²⁺, and change the configuration of liquid crystal droplets.

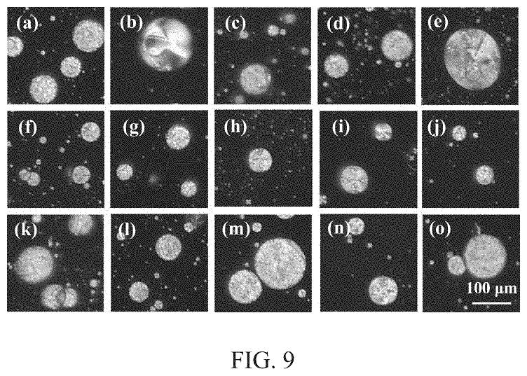

Further, in order to confirm that the liquid crystal droplets containing ZT in the ADLC system are only selective for Hg²⁺, in present invention, a solution containing a different metal ion is added separately for test, which comprises common Na⁺, K⁺, Mg²⁺, Ca²⁺, and some highly toxic heavy metals such as Pb²⁺, Cd²⁺, CU²⁺, Zn²⁺, and Co²⁺. 15 metal ions are used in total, and the concentration of the metal ion is 500 μM. FIG. 8 shows the analysis of selectivity of the ADLC system of the present invention, in which (a)-(p) represent separately (a) an aqueous solution containing 500 μM Hg²⁺, (b) an aqueous solution containing 500 μM Al³⁺500 μM, (c) an aqueous solution containing 500 μM Fe³⁺, (d) an aqueous solution containing 500 μM V³⁺, (e) an aqueous solution containing 500 μM Cd²⁺, (f) an aqueous solution containing 500 μM Zn²⁺, (g) an aqueous solution containing 500 μM Cu²⁺, (h) an aqueous solution containing 500 μM Pb²⁺, (i) an aqueous solution containing 500 μM Mn²⁺, (j) an aqueous solution containing 500 μM Ni²⁺, (k) an aqueous solution containing 500 μM Co²⁺, (l) an aqueous solution containing 500 μM Mg²⁺, (m) an aqueous solution containing 500 μM Ca²⁺, (n) an aqueous solution containing 500 μM Li²⁺, (o) an aqueous solution containing 500 μM Na⁺, and (p) an aqueous solution containing 500 μM K. The test results show that the liquid crystal droplets will only respond to the solution containing Hg²⁺, and become irregular in configuration; and the remaining ions cannot exert an influence, and the liquid crystal droplets still maintain a radial configuration. Therefore, the present invention confirmed that liquid crystal droplets containing ZT in agarose have extremely high selectivity to Hg²⁺.

In the water quality detection, since the test liquid may contain a plurality of metal ions at the same time, whether other metal ions affect the detection of Hg²⁺ by liquid crystal droplets containing ZT in agarose, is then further tested. FIG. 9 shows the analysis of inference by metals with ADLC of the present invention, in which (a)-(o) represent separately (a) an aqueous solution containing 500 μM Hg²⁺ and 500 μM Al³⁺, (b) an aqueous solution containing 500 μM Hg²⁺ and 500 μM Fe³⁺, (c) an aqueous solution containing 500 μM Hg²⁺ and 500 μM V³⁺, (d) an aqueous solution containing 500 μM Hg²⁺, and 500μM Cd²⁺, (e) an aqueous solution containing 500 μM Hg²⁺ and 500 μM Zn²⁺, (f) an aqueous solution containing 500 μM Hg²⁺ and 500 μM Cu²⁺, (g) an aqueous solution containing 500 μM Hg²⁺ and 500 μM Pb²⁺, (h) an aqueous solution containing 500 μM Hg²⁺ and 500 μM Mn²⁺, (i) an aqueous solution containing 500 μM Hg²⁺ and 500 μM Ni²⁺, (j) an aqueous solution containing 500 μM Hg²⁺ and 500 μM Co²⁺, (k) an aqueous solution containing 500 μM Hg²⁺ and 500 μM Mg²⁺, (l) an aqueous solution containing 500 μM Hg²⁺ and 500 μM Ca²⁺, (m) an aqueous solution containing 500 μM Hg²⁺ and 500 μM Li⁺, (n) an aqueous solution containing 500 μM Hg²⁺ and 500 μM Na⁺, and (o) an aqueous solution containing 500 μM Hg²⁺ and 500 μM K⁺. The test results show that when the test solution contains another metal ion, the detection of Hg²⁺ by the liquid crystal droplets containing ZT in the agarose is not affected, and the liquid crystal droplets will still exhibit the irregular configuration. Therefore, the ADLC system can be used in the detection of quality of water with complex pollution.

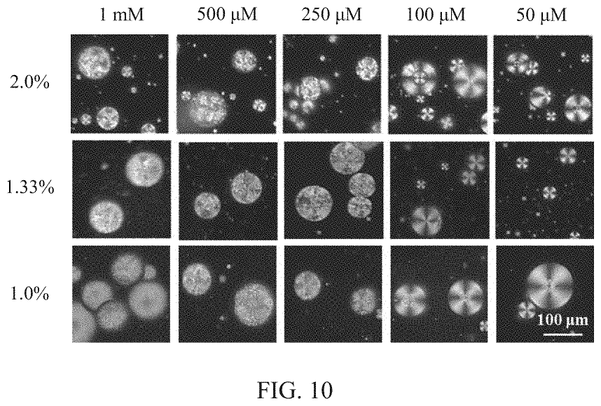

In the present invention, the detection limit of Hg²⁺ concentration by the agarose film containing liquid crystal droplets of ADLC is further tested. FIG. 10 shows the analysis of detection limits for Hg²⁺ by ADLC in the presence of different concentrations of agarose in the present invention. Different concentrations of agarose were prepared by dissolving 1 g of agarose powder in 50 ml, 75 ml and 100 ml of ultrapure water (where the concentration was 2.0% (w/w), 1.33% (w/w), and 1.0% (w/w), respectively). The test results showed that the detection limit for Hg²⁺ was 250 μM in the presence of the three concentrations.

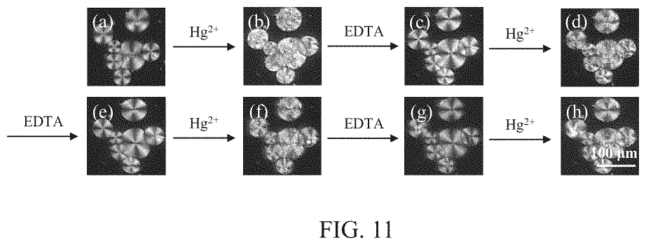

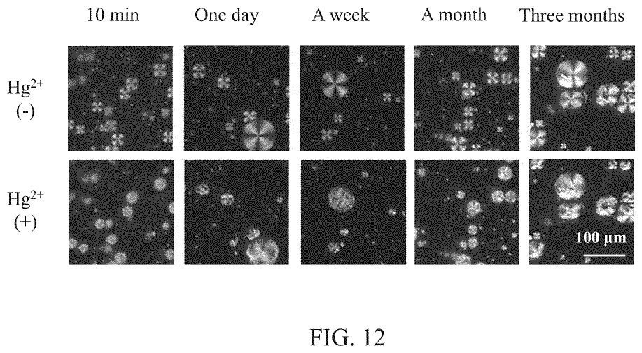

Finally, in order to confirm that the agarose film containing liquid crystal droplets of ADLC provided by the present invention has reversibility and stability, the following tests were conducted in the present invention. FIG. 11 shows the analysis of reuse rate of the ADLC system of the present invention. FIG. 12 is a diagram showing the configuration change of liquid crystal droplets in the ADLC system against Hg²⁺ over long time of detection according to the present invention, in which (a)-(e) show the configurations of liquid crystal droplets of the ADLC at various time points, including, (a) 10 min, (b) 1 day, (c) 7 days, (d) 1 month and (e) 3 months. As can be known from the test results in FIG. 11, when 500 μM Hg²⁺ is firstly added to the ADLC film, the liquid crystal droplets change from radial configuration to irregular configuration; after 1 mM EDTA is then added, the configuration changes from irregular to radial. This cycle can be repeated four times. After the fourth addition of Hg²⁺, the configuration change of liquid crystal droplets is not obvious. It is speculated that this may be because the doped ligand ZT molecule is carried away from the liquid crystal droplets with the mercuric ion by EDTA, so that the concentration of the ligand ZT molecule doped in the liquid crystal droplets after the fourth cycle is too low, and the binding to Hg²⁺ is insufficient to make the LCD configuration change obviously.

In addition, it can be seen from the results of FIG. 12 that the configuration of liquid crystal droplets in the ADLC system can be maintained for more than three months in agarose without obvious change, and the radial configuration is still maintained after three-month storage at room temperature. After adding Hg²⁺, the configuration can still be changed to irregular, indicating that the ADLC system of the present invention is stable and the effectiveness can be maintained for up to three months.

What is claimed is:

1. A method for preparing a polymer gel film containing liquid crystal droplets, comprising the steps of:
preparing liquid crystal droplets containing a ligand, by adding a nematic liquid crystal containing a ligand to an aqueous surfactant solution, and mixing for 10-30 sec by agitating at a rotation speed of 3000 rpm, to obtain a solution of the liquid crystal droplets containing a ligand;
adding the solution of the liquid crystal droplets containing a ligand to an aqueous polymer solution, and mixing by agitating at a rotation speed of 3000 rpm, to obtain an aqueous polymer solution containing the liquid crystal droplets, wherein the ratio of the solution of the liquid crystal droplets containing a ligand to the aqueous polymer solution is 1:1; and
spreading the aqueous polymer solution containing the liquid crystal droplets flatly in a polymer container, and allowing the aqueous polymer solution containing the liquid crystal droplets to gelatinize for 10-15 min, to obtain a polymer gel film containing liquid crystal droplets.

2. The method for preparing a polymer gel film containing liquid crystal droplets according to claim 1, wherein the ratio of the aqueous surfactant solution to the nematic liquid crystal containing a ligand is in the range of 100:1-120:1.

3. The method for preparing a polymer gel film containing liquid crystal droplets according to claim 1, wherein the nematic liquid crystal containing a ligand is prepared through a process comprising:
dissolving a ligand powder in a nematic liquid crystal to formulate a nematic liquid crystal containing 0.3-0.5% of the ligand.

4. The method for preparing a polymer gel film containing liquid crystal droplets according to claim 3, wherein the ligand is 5-(pyridine-4-yl)-2-(5-(pyridin-4-yl)-thiophen-2-yl)thiazole (ZT), a ligand having specificity for mercuric ions.

5. The method for preparing a polymer gel film containing liquid crystal droplets according to claim 1, wherein the aqueous surfactant solution is prepared through a process comprising:
dissolving a cationic emulsifier powder in ultrapure water to prepare a mother liquor having a concentration of 1%; and
then diluting the mother liquor into various concentrations of aqueous surfactant solutions, including 0.03%, 0.05%, and 0.005%.

6. The method for preparing a polymer gel film containing liquid crystal droplets according to claim 5, wherein the cationic emulsifier is selected from a group consisting of hexadecyltrimethylammonium bromide (C₁₆TAB), tetradecyltrimethylammonium bromide (C₁₄TAB), dodecyltrimethylammonium bromide (C₁₂TAB), and decyltrimethylammonium bromide (C₁₀TAB).

7. The method for preparing a polymer gel film containing liquid crystal droplets according to claim 1, wherein the aqueous polymer solution is prepared through a process comprising:
dissolving a polymer powder in ultrapure water; and
heating to 80° C. to completely dissolve the polymer powder, to obtain an aqueous polymer solution, wherein the concentration of the aqueous polymer solution was 1-2%.

8. The method for preparing a polymer gel film containing liquid crystal droplets according to claim 7, wherein the aqueous polymer solution is an aqueous agarose solution or aqueous chitosan solution.

9. A polymer gel film containing liquid crystal droplets, prepared by the method for preparing a polymer gel film containing liquid crystal droplets according to claim 1.

10. A method for detecting mercuric ions in an aqueous solution by using the polymer gel film containing liquid crystal droplets according to claim 9, comprising taking 10-30 μL of a test solution;
applying the test solution to the polymer gel film containing liquid crystal droplets; and
locating the polymer gel film containing liquid crystal droplets applied with the test solution under a cross-polarized microscope, and observing the conformation inside the liquid crystal droplets.
